(19) United States
(12) Patent Application Publication
(10) Pub. No.: US 2024/0002943 A1
(43) Pub. Date: Jan. 4, 2024

(54) A METHOD FOR CLASSIFYING THE GUT INFLAMMATION STATUS IN AVIAN SPECIES

(71) Applicant: Evonik Operations GmbH, Essen (DE)

(72) Inventors: Florian BÖHL, Neckargemünd (DE); Frank LYKO, Hirschberg an der Bergstraße (DE); Walter PFEFFERLE, Langgöns (DE); Andreas KAPPEL, Glashütten (DE); Rose WHELAN, Ambrosden, Oxfordshire (GB); Günter RADDATZ, Heidelberg (DE); Monika FLÜGEL, Steinhagen (DE); Stefan PELZER, Gütersloh (DE); Achim MARX, Gelnhausen (DE); Emery STEPHANS, Stamford, CT (US); Frank THIEMANN, Nottuln (DE); Emeka Ignatius IGWE, München (DE)

(73) Assignee: Evonik Operations GmbH, Essen (DE)

(21) Appl. No.: 18/265,029

(22) PCT Filed: Nov. 22, 2021
(86) PCT No.: PCT/EP2021/082444

§ 371 Date: Jun. 2, 2023

(30) Foreign Application Priority Data
Dec. 4, 2020 (EP) 20211764.4

(51) Int. Cl. C12Q 1/6883; G16B 40/00

(52) U.S. Cl. CPC C12Q 1/6883; G16B 40/00; C12Q 2600/154

(57) ABSTRACT

The invention provides a method for classifying the gut inflammation status of an avian subject or of a group of avian subjects to be tested, the method comprising the comparison of the average methylation levels within a panel of pre-selected LMRs in the genomic DNA isolated from gut sample material deriving from the individual avian subject or of the group of avian subjects to be tested with the average methylation levels of the same panel of LMRs in the genomic DNA pertaining to one or more reference samples having a negative gut inflammation status.

### FIELD OF THE INVENTION

The invention pertains to an epigenetics-based method for classifying the gut inflammation status and to a method for developing a test system for classifying the gut inflammation status of avian gut samples, respectively.

### BACKGROUND OF THE INVENTION

Intestinal health is critically important for the welfare and performance of livestock animals, and in particular for avian species, such as poultry/chicken. Enteric diseases and inflammatory processes that affect the structural integrity of the gastrointestinal tract (GIT) lead to high economic losses due to reduced weight gain, poor feed conversion efficiency, increased mortality rates and greater medication costs.

An intact intestinal barrier provides a number of physiological and functional features, including nutrient digestion and absorption, host metabolism and energy generation, a stable microbiome, mucus layer development, barrier function, and mucosal immune responses. As the largest organ in the body, the gut serves as a selective barrier to take up nutrients and fluids into the body, while excluding undesirable molecules and pathogens. Therefore, proper gut function is essential to maintain optimal health and balance throughout the body and represents a key line of defense against foreign antigens from the environment.

Recently, there has been increased interest in research on intestinal permeability in chickens, resulting in different strategies to measure intestinal inflammation and concomitant intestinal barrier failure. However, none of the proposed strategies represent a good marker for the broiler industry as they are either not applicable under field conditions or non-specific for intestinal inflammation.

Accordingly, a marker, or a set of markers, that can accurately detect intestinal inflammation and concomitant perturbation of the intestinal integrity in avian species would be highly desirable.

“Epigenetic changes”, i.e. changes in DNA methylation patterns occur naturally but can also be influenced by several factors including age, the environment/lifestyle, and disease state. DNA methylation is a heritable change in phenotype that regulates gene expression without a change in genotype and thus does not involve changes to the underlying DNA sequence, but instead acts through the chemical modification of the DNA by methylation of CpG dinucleotides (5′-C-phosphate-G-3′), i.e. regions of DNA where a cytosine nucleotide is followed by a guanine nucleotide in the linear sequence of bases along its 5′→3′ direction. Depending on the age and/or environment, many organisms develop tissue-specific methylation patterns that are interpunctuated by CpG-islands and canyons (low/no methylation, often associated with promoter regions) and Low-Methylated Regions (LMRs). LMRs represent a key feature of the dynamic methylome and usually coincide with regions or sites of transcription factor binding, which could be occupied or not in response to an environmental factor. Environmental influences changing the epigenetic pattern are e.g. diet, disease, microbiome, temperature, stress. Therefore, these methylation patterns display the interaction of a body/tissue with its environment and, as such, are a suitable readout to evaluate at a molecular level the age or health status of a test subject vs. a control group.

In view of the above, it was the objective of the present invention to provide epigenetic markers that enable to accurately and reliably detect intestinal inflammation in avian subjects or groups of subjects.

### SUMMARY OF THE INVENTION

The above objective has been solved by the method according to the present invention. More specifically, the invention pertains to a method for classifying the gut inflammation status of an avian subject or of a group of avian subjects to be tested, the method comprising the comparison of the average methylation levels within a panel of pre-selected LMRs in the genomic DNA isolated from gut sample material deriving/derived from the individual avian subject or of the group of avian subjects to be tested with the average methylation levels of the same panel of LMRs in the genomic DNA pertaining to one or more reference samples having a negative gut inflammation status.

In another aspect of the present invention, there is provided a method for classifying gut inflammation status of an avian subject or of a group of avian subjects to be tested, an avian test subject, the method comprising:
a. determining a test average methylation level of a panel of pre-selected Low-Methylated Regions (LMRs) in isolated genomic DNA from a gut test sample derived from the avian test subject;
b. comparing the test average methylation level obtained in step (a) with a reference average methylation level of the same panel of LMRs in genomic DNA isolated from at least one avian gut sample having a negative gut inflammation status,
   wherein when the test average methylation level is substantially similar to the reference average methylation level, then the test subject has a negative gut inflammation status and when the test average methylation level is different from the reference average methylation level then the test subject has a positive gut inflammation status.

The term “significantly similar” in the context of the disclosure is a similarity observed by either statistical means (i.e. bioinformatics) or by empiric observation.

In another aspect of the present invention, there is provided a method for developing a test system (“classifier”) for classifying the gut inflammation status of avian gut samples. In particular, the method comprises the steps of
a. detecting Low-Methylated Regions (LMRs) in genomic DNA in gut sample obtained from an avian subject or from a group of avian subjects with a known gut inflammation status,
b. selecting a panel of LMRs from the LMRs of step (a) suitable for each known gut inflammation status, such that the classification reliability of the selected panel of LMRs is optimized for the assignment of the known gut inflammation status,
c. measuring the average methylation levels of the selected panel of LMRs for each known gut inflammation status, and
d. creating a library of different reference average methylation levels of the selected panel of LMRs for respective known gut inflammation status,
   wherein a comparison of an average methylation level obtained from a gut test sample with the reference average methylation levels of the selected panel of LMRs allows for classifying the gut inflammation status of the gut test sample.

### DETAILED DESCRIPTION OF THE INVENTION

In the following, the elements of the invention will be described. The terms “of the [present] invention”, “in accordance with the invention”, “according to the invention” and the like, as used herein are intended to refer to all aspects and embodiments of the invention described and/or claimed herein. As used herein, the term “comprising” is to be construed as encompassing both “including” and “consisting of”, both meanings being specifically intended, and hence individually disclosed embodiments in accordance with the present invention.

Unless context dictates otherwise, the descriptions and definitions of the features set out above are not limited to any particular aspect or embodiment of the invention and apply equally to all aspects and embodiments which are described.

The term “methylation level” refers to the level of a specific methylation site which can range from 0 (=unmethylated) to 1 (=fully methylated). Thus, based on the methylation level of one or more methylation sites, a methylation profile may be determined. Accordingly, the term “methylation” profile” or also “methylation pattern” refers to the relative or absolute concentration of methylated C or unmethylated C at any particular stretch of residues in a biological sample. For example, if cytosine (C) residue(s) not typically methylated within a DNA sequence are more methylated in a sample, it may be referred to as “hypermethylated”; whereas if cytosine (C) residue(s) typically methylated within a DNA sequence are less methylated, it may be referred to as “hypomethylated”. Likewise, if the cytosine (C) residue(s) within a DNA sequence (e.g., sample nucleic acid) are more methylated when compared to another sequence from a different region or from a different individual (e.g., relative to normal nucleic acid), that sequence is considered hypermethylated compared to the other sequence. Alternatively, if the cytosine (C) residue(s) within a DNA sequence are less methylated as compared to another sequence from a different region or from a different individual, that sequence is considered hypomethylated compared to the other sequence. These sequences are said to be “differentially methylated”. For example, when the methylation status differs between inflamed and non-inflamed tissues, the sequences are considered “differentially methylated. Measurement of the levels of differential methylation may be done by a variety of ways known to those skilled in the art. One method is to measure the methylation level of individual interrogated CpG sites determined by the bisulfite sequencing method, as a non-limiting example.

As used herein, a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring, however, for purposes herein, thymine is not considered a methylated nucleotide when present in DNA since thymine is a typical nucleotide base of DNA. Typical nucleoside bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated.

As used herein, a “CpG site” or “methylation site” is a nucleotide or sequence of nucleotides within a nucleic acid that is susceptible to methylation either by natural occurring events in vivo or by an event instituted to chemically methylate the nucleotide in vitro.

As used herein, a “methylated nucleic acid molecule” refers to a nucleic acid molecule that contains one or more nucleotides that is/are methylated.

A “CpG island” as used herein describes a segment of DNA sequence with elevated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745).

The term “bisulfite” as used herein encompasses any suitable type of bisulfite, such as sodium bisulfite, or other chemical agent that is capable of chemically converting a cytosine (C) to a uracil (U) without chemically modifying a methylated cytosine and therefore can be used to differentially modify a DNA sequence based on the methylation status of the DNA, e.g., U.S. Pat. Pub. US 2010/0112595 (Menchen et al.). As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

In the context of testing for a methylation status at any given methylation site the invention comprises also any “non-bisulfite-based method” and “non-bisulfite-based quantitative method” Such terms refer to any method for quantifying a methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acids using agents that bind nucleic acids based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

The inventors have unexpectedly found that the inflammation status of avian gut material can be successfully classified by comparing the average methylation levels within a panel of pre-selected LMRs in the genomic DNA isolated from a test gut sample with the average methylation levels of the same panel of LMRs pertaining to one or more reference samples having a negative gut inflammation status. The term “negative gut inflammation status” refers to gut material free of any signs of inflammatory processes.

In the context of the present invention, the term “gut inflammation” and “intestinal inflammation” are used interchangeably and have identical meanings. The expression “classifying the gut inflammation status” refers to both, the classification whether or not there are inflammatory processes ongoing (YES/NO), as well as to the classification into different inflammation grades (e.g. severe, moderate or weak inflammations). In particular, the positive gut inflammation status is further classified into classes of severely inflamed, moderately inflamed, or weakly inflamed based on the average methylation level.

In a particular aspect of the present invention, the method comprises the steps of
a. isolating the genomic DNA from a gut sample of the avian subject or of the avian population to be tested,
b. determining the average methylation level of a panel of pre-selected LMRs in the genomic DNA obtained in step (a.),
c. comparing the average methylation level of a panel of pre-selected LMRs obtained in step (b) with the average methylation level of the same panel of LMRs within the genomic DNA pertaining to one or more reference samples having a negative gut inflammation status,
   wherein if the average methylation level of the panel LMRs of the test sample is significantly similar to one of the one or more predetermined reference samples, the gut inflammation status if the avian subject or of the avian population to be tested is similar to the inflammation status of the respective reference sample.

In particular, the method comprises the steps of:
a. determining a test average methylation level of a panel of pre-selected Low-Methylated Regions (LMRs) in isolated genomic DNA from a gut test sample derived from the avian test subject;
b. comparing the test average methylation level obtained in step (a) with a reference average methylation level of the same panel of LMRs in genomic DNA isolated from at least one avian gut sample having a negative gut inflammation status,
   wherein when the test average methylation level is substantially similar to the reference average methylation level, then the test subject has a negative gut inflammation status and when the test average methylation level is different from the reference average methylation level then the test subject has a positive gut inflammation status.

As used herein, the term “panel of pre-selected LMRs” refers to a panel of LMRs having CpGs that show a strand specific coverage of equal or greater than 5. Further, CpG sites known as single nucleotide polymorphisms (SNPs) may be filtered out. In particular, any method known in the art may be used to identify or detect LMRs in the genomic DNA. Well known methods include using programmes such as MethylSeekR. In particular, LMRs in the genomic DNA have at least three consecutive CpGs and have no single nucleotide polymorphisms (SNPs) in any of the CpG positions. Further, the LMR is a region of the genome wherein less than 60% of CpGs in that region are methylated. More in particular, less than 50%, 40%, 30%, 20% or 10% of the CpGs in the LMRs are methylated. Even more in particular, LMRs in the genomic DNA are identified based on the method disclosed at least in Burger, L., (2013) Nucleic Acids Research, 41 (16): e155 and/or Stadler, M., (2011) Nature 480, 490-495. LMRs are known to have an average methylation ranging from 10% to 50%; are regions of low CG density; tend to be enriched for H3K4me1, DHSs, and p300/CBP; and/or are primarily located distal to promoters in intergenic or intronic regions. In particular, LMRs:
have an average methylation ranging from 10% to 50%,
are regions of low CG density;
are enriched for Histone H3 monomethylated at lysine 4 (H3K4me1), DNase I hypersensitive sites (DHSs) and transcriptional coactivators CREB binding protein (CPB) and p300;
are primarily located distal to promoters in intergenic or intronic regions; and/or
have no single nucleotide polymorphisms (SNPs) in any of the CpG positions.

Once the LMRs of the genomic DNA are identified, the panel of LMRs may be selected. The panel of LMRs may be selected using any method known in the art. In particular, the same panel of LMRs may be used to classify avian gut inflammation. That is to say, the average methylation level of the same panel of LMRs may be used to classify if the test sample has positive or negative gut inflammation and further classify if the positively inflamed gut is severely, moderately or weakly inflamed.

In one example, the panel of LMRs used according to any aspect of the present invention may be finally selected using machine learning techniques, such as random forests (Breiman L (2001). “Random Forests”. Machine Learning. 45 (1): 5-32. doi:10.1023/A:1010933404324). The machine-learning system, which advantageously is the random forest predictor, learns the gut inflammation status of individualized animals based on the average methylation values within the genomic LMRs. Said process is further illustrated in the exemplary section.

The panel of LMRs are advantageously pre-selected such that the classification reliability is optimized for the assignment of different inflammation status classes.

The panel of pre-selected LMRs may be optimized for accuracy using the machine learning technique like Random Forest Analysis. In particular, the panel of pre-selected LMRs is at least two LMRs selected from the following list of LMRs of LMR1 to LMR15:

| LMR | chromosome | Start | End |
|---|---|---|---|
| 1 | chr1 | 4035842 | 4036031 |
| 2 | chr1 | 194933340 | 194933476 |
| 3 | chr2 | 25436290 | 25437730 |
| 4 | chr2 | 146200788 | 146200966 |
| 5 | chr3 | 27449524 | 27449646 |
| 6 | chr4 | 2434473 | 2434747 |
| 7 | chr5 | 33274718 | 33277003 |
| 8 | chr7 | 2418236 | 2418612 |
| 9 | chr11 | 4103942 | 4104386 |
| 10 | chr12 | 3121599 | 3121777 |
| 11 | chr12 | 16120842 | 16120882 |
| 12 | chr13 | 8094316 | 8094824 |
| 13 | chr13 | 14903544 | 14903714 |
| 14 | chr15 | 7910436 | 7910504 |
| 15 | chr21 | 5370979 | 5371156 |

More in particular, the panel of pre-selected LMRs is at least 3, 4, 5, 6, 7, 8, 9, 10, 11, 12, 13, 14 or 15 LMRs selected from the list of LMRs of LMR1 to LMR15. Even more in particular, the panel of pre-selected LMRs is at least 12, LMRs selected from the list of LMRs of LMR1 to LMR15.

In another example, the panel of LMRs may be selected based on other methods such as maximum methylation difference between the LMRs from genomic DNA from at least one avian gut sample having a negative gut inflammation status, and LMRs from genomic DNA from at least one avian gut sample having a positive gut inflammation status. It is well within the common general knowledge of a skilled person to carry out these methods. In particular, the panel of pre-selected LMRs is at least two LMRs selected from the following list of LMRs of LMR16 to LMR30:

| LMR | chromosome | Start | End |
|---|---|---|---|
| 16 | chr1 | 18054888 | 18054950 |
| 17 | chr1 | 40284190 | 40285196 |
| 18 | chr2 | 29647196 | 29647230 |
| 19 | chr2 | 31991770 | 31991962 |
| 20 | chr2 | 55880962 | 55880988 |
| 21 | chr2 | 121135192 | 121135560 |
| 22 | chr3 | 67444230 | 67444652 |
| 23 | chr3 | 101470002 | 101470054 |
| 24 | chr4 | 2049172 | 2049946 |
| 25 | chr4 | 7321072 | 7321506 |
| 26 | chr6 | 16995134 | 16995161 |
| 27 | chr12 | 1423884 | 1424138 |
| 28 | chr13 | 2182022 | 2182138 |
| 29 | chr14 | 4612818 | 4613149 |
| 30 | chr14 | 12751458 | 12751702 |

More in particular, the panel of pre-selected LMRs is at least 3, 4, 5, 6, 7, 8, 9, 10, 11, 12, 13, 14 or 15 LMRs selected from the list of LMRs of LMR16 to LMR30. Even more in particular, the panel of pre-selected LMRs is at least 12, LMRs selected from the list of LMRs of LMR16 to LMR30.

The step of determining the average methylation levels within a panel of pre-selected LMRs in the genomic DNA isolated from gut sample material deriving from the individual avian subject or of the group of avian subjects to be tested may include bisulfite conversion process. Therein, cytosine residues in the genomic DNA are transformed to uracil, while 5-methylcytosine residues in the genomic DNA are not transformed to uracil.

Whole genome bisulfite sequencing is a genome-wide analysis of DNA methylation based on the sodium bisulfite conversion of genomic DNA, which is then sequenced on a next-generation sequencing platform. The sequences are then aligned to the reference genome to determine methylation states of the CpG dinucleotides based on mismatches resulting from the conversion of unmethylated cytosines into uracil.

For example, methylation levels can be measured using commercial Illumina™ sequencing or array platforms.

The avian subject or the group of avian subjects to be tested may be poultry, such as chickens, turkeys, ducks and geese. Preferably, the avian subject or the group of avian subjects to be tested is chicken(s).

The gut sample material may be gut tissue, said gut tissue preferably being ileum or jejunum.

Alternatively, the gut sample material may be a sample material comprising gut mucosa, such as feces.

In a particular example, the gut sample material is a pooled sample deriving from a group of avian subjects to be tested.

According to another aspect of the present invention, there is provided a method for developing a test system for classifying the gut inflammation status of avian gut samples, the method comprising
a. providing one or more gut samples of obtained from avian subject or from a group of avian subjects with a known gut inflammation status,
b. determining the average methylation levels of one or more LMRs within the genomic DNA contained in each of the one or more gut sample obtained in step (a),
c. selecting from the one or more LMRs of step (b) a panel of LMRs such that the classification reliability of the panel LMRs is optimized for the assignment of each known inflammation status and
d. assigning a panel LMR reference methylation profile for each known gut inflammation status,
   wherein a comparison of the average methylation levels obtained from a gut test sample with the panel LMR reference methylation profile allows for classifying the gut inflammation status of the gut test sample.

In particular, there is provided a method for developing a test system for classifying gut inflammation status of avian gut samples, the method comprising
a. detecting Low-Methylated Regions (LMRs) in genomic DNA in gut sample obtained from an avian subject or from a group of avian subjects with a known gut inflammation status,
b. selecting a panel of LMRs from the LMRs of step (a) suitable for each known gut inflammation status, such that the classification reliability of the selected panel of LMRs is optimized for the assignment of the known gut inflammation status,
c. measuring the average methylation levels of the selected panel of LMRs for each known gut inflammation status, and
d. creating a library of different reference average methylation levels of the selected panel of LMRs for respective known gut inflammation status,
   wherein a comparison of an average methylation level obtained from a gut test sample with the reference average methylation levels of the selected panel of LMRs allows for classifying the gut inflammation status of the gut test sample.

In particular, the library of different reference average methylation levels is based on a single panel of LMRs with different reference average methylation levels dependent on whether the reference sample had positive or negative gut inflammation and further whether the positive gut inflammation is severe, moderate or weak.

### BRIEF DESCRIPTION OF THE FIGURES

FIG. 1: Classification error of an iterative random forest 3-fold cross-validation with 100 LMRs as starting input, applying the command rfcv of the R package RandomForest. The figure shows that the error becomes very small starting at 13 LMRs. At 6 LMRs the predictive power decreases and the error increases significantly.

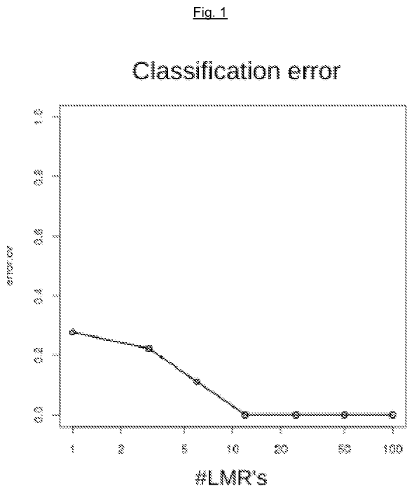

FIG. 2: Establishment of a chicken DNA methylation random forest classifier of the gut inflammation status as described in the exemplary section.

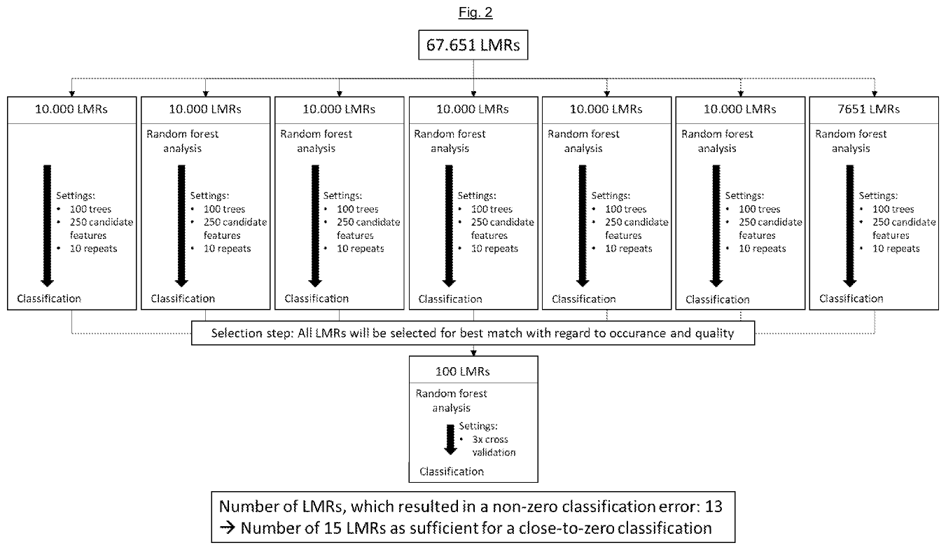

### EXAMPLES

Certain aspects and embodiments of the invention will now be illustrated byway of example and with reference to the description, figures and tables set out herein. Such examples of the methods, use and other aspects of the present invention are representative only, and should not be taken to limit the scope of the present invention to only such representative examples.

Methods

A broiler study was conducted with Ross 308 male broilers fed industry standard, three phase, corn-soybean meal diets formulated to meet all nutrient requirements from day 1-35 (Table 1).

TABLE 1

| | Starter | Grower | Finisher |
|---|---|---|---|
| Ingredients, % | (day 1-14) | (day 15-28) | (day 29-35) |
| Corn | 54.38 | 62.93 | 63.24 |
| Soybean Meal (48% CP) | 35.00 | 26.83 | 25.48 |
| Corn Gluten Meal (60% CP) | 4.00 | 4.00 | 4.00 |
| Soybean Oil | 2.66 | 2.45 | 3.80 |
| Dicalcium phosphate 22 | 1.74 | 1.61 | 1.42 |
| Limestone (CaCO₃) | 0.75 | 0.75 | 0.69 |
| Salt (NaCl) | 0.36 | 0.37 | 0.34 |
| Choline Chloride 60% | 0.10 | 0.10 | 0.10 |
| Vitamin Mineral Premix | 0.50 | 0.50 | 0.50 |
| DL-Methionine | 0.25 | 0.20 | 0.21 |
| L-Lysine-HCl | 0.22 | 0.23 | 0.19 |
| L-Threonine | 0.04 | 0.04 | 0.03 |
| Total | 100 | 100 | 100 |
| Nutrient composition, as is | | | |
| ME, kcal/kg | 3008 | 3086 | 3186 |
| CP, % | 23.90 | 20.45 | 19.69 |
| Ca | 0.90 | 0.84 | 0.76 |
| Available Phosphorous | 0.45 | 0.42 | 0.38 |
| Lysine | 1.36 | 1.15 | 1.09 |
| Methionine | 0.62 | 0.53 | 0.52 |
| Methionine + Cysteine | 1.00 | 0.86 | 0.84 |
| Threonine | 0.9, | 0.80 | 0.76 |
| Tryptophan | 0.27 | 0.23 | 0.21 |
| Arginine | 1.50 | 1.30 | 1.18 |
| Isoleucine | 1.00 | 0.85 | 0.80 |
| Leucine | 2.19 | 1.98 | 1.91 |
| Valine | 1.10 | 0.95 | 0.90 |

Three physiologically healthy birds were euthanized each at days 3, 15 and 35 to excise spleen, intestinal (ileum) and muscle (pectoralis major) samples for DNA extraction (an Invitrogen PureLink genomic DNA isolation kit) and bisulfite sequencing.

Samples

The animals were stratified into two groups (inflamed and non-inflamed with three time points each). From each of these 2 groups, DNA was prepared from nine independent animals, resulting in 18 genomic DNA samples.

Whole-Genome Bisulfite Sequencing

Libraries were prepared using the Accel-NGS Methyl-Seq DNA Library Kit from Swift Biosciences. Two sequencing libraries were barcoded onto one sequencing lane. Sequencing was performed on an Illumina HiSeq X platform using a standard paired-end sequencing protocol with 105 nucleotides read length.

TABLE 2
Whole-genome bisulfite sequencing datasets generated in this study

| Tissue | age (days) | no. of samples | coverage | conversion |
|---|---|---|---|---|
| Jejunum | 14 | 3 | 55.4x | >99.9% |
| | 16 | 3 | 54.7x | >99.9% |
| | 34 | 3 | 57.7x | >99.9% |
| Jejunum | 14 | 3 | 43.3x | >99.9% |
| | 16 | 3 | 47.4x | >99.9% |
| | 34 | 3 | 46.7x | >99.9% |

Read Mapping

Reads were trimmed and mapped with BSMAP 2.5 (Xi Y, Li W. 2009. BSMAP: whole genome bisulfite sequence MAPping program. BMC Bioinformatics 10:232. doi:10.1186/1471-2105-10-232.) using the Gallus gallus genome assembly version 5.0 (https://www.ebi.ac.uk/ena/data/view/GCA_000002315.3) as a reference sequence. Duplicates were removed using the Picard tool (http://broadinstitute.github.io/picard). Methylation levels were determined using a Python script (methratio.py) distributed together with the BSMAP package by dividing the number of reads having a methylated CpG at a certain genomic position by the number of all reads covering this position.

SNP Filtering of the Methylation Data

All CpGs which are listed as SNPs in the database dbSNP for the Gallus gallus genome were filtered out. For the LMR based random forest, the analysis was restricted to CpGs within low-methylated regions that showed a strand specific coverage of greater than 5 in every of the sequenced samples, resulting in a set of 67,651 LMRs.

Establishment of a chicken DNA methylation random forest classifier of the gut inflammation status A random forest predictor (implemented in the R package randomForest [https://cran.r-project.org/web/packages/randomForest/]) was applied to learn the gut inflammation status of the animals, based on the average methylation values of the LMRs. This was done by splitting the whole set of LMRs in chunks of 10.000 LMRs and using each chunk as input for the algorithm by fitting 100 trees using 250 candidate features (LMRs) at each split. All LMRs from each chunk which showed a value greater than zero for the variable importance measure “decrease_accuracy”, were pooled. This was repeated 10 times and for every LMR it was counted how often it was found in the pooled sets of these 10 repetitions. The 100 LMRs with the most occurrences were kept. Then an iterative random forest 3-fold cross-validation with these 100 LMRs as input was performed, applying the command rfcv of the package RandomForest, which stepwise decreases the number of features used as input. The resulting classification error was recorded and the number of LMRs, which resulted in a non-zero classification error was evaluated. This value was found to be 13 LMRs. We decided to consider a number of 15 LMRs as sufficient for a close-to-zero classification. The 15 LMRs with the highest value of “decrease_accuracy” are considered to be the resulting feature set (table 3).

In a second step, the average methylation difference of all LMRs between control and inflamed samples was evaluated and the 15 LMRs showing the highest average methylation difference were kept (table 4).

TABLE 3
RF LMRs, #LMR's: 15

| ID | chromosome | Start | End |
|---|---|---|---|
| 1 | chr1 | 4035842 | 4036031 |
| 2 | chr1 | 194933340 | 194933476 |
| 3 | chr2 | 25436290 | 25437730 |
| 4 | chr2 | 146200788 | 146200966 |
| 5 | chr3 | 27449524 | 27449646 |
| 6 | chr4 | 2434473 | 2434747 |
| 7 | chr5 | 33274718 | 33277003 |
| 8 | chr7 | 2418236 | 2418612 |
| 9 | chr11 | 4103942 | 4104386 |
| 10 | chr12 | 3121599 | 3121777 |
| 11 | chr12 | 16120842 | 16120882 |
| 12 | chr13 | 8094316 | 8094824 |
| 13 | chr13 | 14903544 | 14903714 |
| 14 | chr15 | 7910436 | 7910504 |
| 15 | chr21 | 5370979 | 5371156 |

TABLE 4
LMRs, optimized for effect size, #LMR's: 15

| ID | chromosome | Start | End |
|---|---|---|---|
| 1 | chr1 | 18054888 | 18054950 |
| 2 | chr1 | 40284190 | 40285196 |
| 3 | chr2 | 29647196 | 29647230 |
| 4 | chr2 | 31991770 | 31991962 |
| 5 | chr2 | 55880962 | 55880988 |
| 6 | chr2 | 121135192 | 121135560 |
| 7 | chr3 | 67444230 | 67444652 |
| 8 | chr3 | 101470002 | 101470054 |
| 9 | chr4 | 2049172 | 2049946 |
| 10 | chr4 | 7321072 | 7321506 |
| 11 | chr6 | 16995134 | 16995161 |
| 12 | chr12 | 1423884 | 1424138 |
| 13 | chr13 | 2182022 | 2182138 |
| 14 | chr14 | 4612818 | 4613149 |
| 15 | chr14 | 12751458 | 12751702 |

Table 5 is an example for a trained random forest, using the 15 LMRs from table 3, constructed with the command “randomForest” of the R-package randomForest. As input data, the methylation values of the 15 LMRs for the 9 con (control) samples and the 9 infl (inflamed) samples were used, each value computed as the average over the whole LMR. The forest contains 100 trees and for every split 4 LMRs were used (parameter mtry). This forest was selected, as it classified all 18 samples correctly as control or inflamed, based on the internal validation using COB (out-of-bag) data.

Depending on the LMR methylation values of every sample the tree is travelled along, until a terminal node is reached. This terminal node is assigned to either “con” (control) or “infl” (inflamed) and in this way the classification is performed. Consequently, a non-terminal node contains “NA” (not applicable) in the “prediction” column, as reaching it does not allow a classification of the sample at this point, which is only possible, when a terminal node is reached. For each line in the table applies:
Field 1 (left_daughter): Number of left daughter node; value is 0, if there are none
Field 2 (right_daughter): Number of right daughter node; value is 0, if there are none
Field 3 (split_var): LMR that is used for the decision, which daughter-node is selected; not applicable (NA) for terminal nodes
Field 4 (split_point): Numerical value of the “split_var” which has to be fallen below (for selecting the left daughter node) or exceeded (for selecting the right daughter node).
Field 5 (Status): Terminal node (−1) or non-terminal nodes (1)
Field 6: (prediction): Classification in control (con) or inflamed (infl) of terminal node; NA, if node is not terminal

TABLE 5

| Tree | left_daughter | right_daughter | split_var | split_point | status | prediction |
|---|---|---|---|---|---|---|
| 1 | 2 | 3 | LMR9 | 0.235 | 1 | NA |
| | 0 | 0 | NA | 0 | −1 | infl |
| | 0 | 0 | NA | 0 | −1 | con |
| 2 | 2 | 3 | LMR2 | 0.055 | 1 | NA |
| | 0 | 0 | NA | 0 | −1 | con |
| | 0 | 0 | NA | 0 | −1 | infl |
| 3 | 2 | 3 | LMR12 | 0.356 | 1 | NA |
| | 0 | 0 | NA | 0 | −1 | infl |
| | 0 | 0 | NA | 0 | −1 | con |
| 4 | 2 | 3 | LMR8 | 0.446 | 1 | NA |
| | 0 | 0 | NA | 0 | −1 | con |
| | 0 | 0 | NA | 0 | −1 | infl |
| 5 | 2 | 3 | LMR7 | 0.412 | 1 | NA |
| | 0 | 0 | NA | 0 | −1 | con |
| | 0 | 0 | NA | 0 | −1 | infl |
| 6 | 2 | 3 | LMR12 | 0.346 | 1 | NA |
| | 0 | 0 | NA | 0 | −1 | infl |
| | 0 | 0 | NA | 0 | −1 | con |
| 7 | 2 | 3 | LMR5 | 0.191 | 1 | NA |
| | 0 | 0 | NA | 0 | −1 | con |
| | 0 | 0 | NA | 0 | −1 | infl |
| 8 | 2 | 3 | LMR8 | 0.451 | 1 | NA |
| | 0 | 0 | NA | 0 | −1 | con |
| | 0 | 0 | NA | 0 | −1 | infl |
| 9 | 2 | 3 | LMR6 | 0.129 | 1 | NA |
| | 0 | 0 | NA | 0 | −1 | con |
| | 4 | 5 | LMR10 | 0.019 | 1 | NA |
| | 0 | 0 | NA | 0 | −1 | infl |
| | 0 | 0 | NA | 0 | −1 | con |
| 10 | 2 | 3 | LMR4 | 0.12 | 1 | NA |
| | 4 | 5 | LMR11 | 0.119 | 1 | NA |
| | 0 | 0 | NA | 0 | −1 | con |
| | 0 | 0 | NA | 0 | −1 | con |
| | 0 | 0 | NA | 0 | −1 | infl |
| 11 | 2 | 3 | LMR6 | 0.12 | 1 | NA |
| | 0 | 0 | NA | 0 | −1 | con |
| | 4 | 5 | LMR7 | 0.39 | 1 | NA |
| | 0 | 0 | NA | 0 | −1 | con |
| | 0 | 0 | NA | 0 | −1 | infl |
| 12 | 2 | 3 | LMR12 | 0.358 | 1 | NA |
| | 0 | 0 | NA | 0 | −1 | infl |
| | 0 | 0 | NA | 0 | −1 | con |
| 13 | 2 | 3 | LMR10 | 0.017 | 1 | NA |
| | 0 | 0 | NA | 0 | −1 | infl |
| | 0 | 0 | NA | 0 | −1 | con |
| 14 | 2 | 3 | LMR7 | 0.395 | 1 | NA |
| | 0 | 0 | NA | 0 | −1 | con |
| | 0 | 0 | NA | 0 | −1 | infl |
| 15 | 2 | 3 | LMR15 | 0.109 | 1 | NA |
| | 0 | 0 | NA | 0 | −1 | con |
| | 0 | 0 | NA | 0 | −1 | infl |
| 16 | 2 | 3 | LMR4 | 0.115 | 1 | NA |
| | 0 | 0 | NA | 0 | −1 | infl |
| | 0 | 0 | NA | 0 | −1 | con |
| 17 | 2 | 3 | LMR10 | 0.017 | 1 | NA |
| | 4 | 5 | LMR13 | 0.066 | 1 | NA |
| | 0 | 0 | NA | 0 | −1 | con |
| | 0 | 0 | NA | 0 | −1 | con |
| | 0 | 0 | NA | 0 | −1 | infl |
| 18 | 2 | 3 | LMR2 | 0.055 | 1 | NA |
| | 0 | 0 | NA | 0 | −1 | con |
| | 0 | 0 | NA | 0 | −1 | infl |
| 19 | 2 | 3 | LMR10 | 0.016 | 1 | NA |
| | 0 | 0 | NA | 0 | −1 | infl |
| | 0 | 0 | NA | 0 | −1 | con |
| 20 | 2 | 3 | LMR7 | 0.412 | 1 | NA |
| | 0 | 0 | NA | 0 | −1 | con |
| | 0 | 0 | NA | 0 | −1 | infl |
| 21 | 2 | 3 | LMR15 | 0.077 | 1 | NA |
| | 0 | 0 | NA | 0 | −1 | con |
| | 0 | 0 | NA | 0 | −1 | infl |
| 22 | 2 | 3 | LMR9 | 0.235 | 1 | NA |
| | 0 | 0 | NA | 0 | −1 | infl |
| | 0 | 0 | NA | 0 | −1 | con |
| 23 | 2 | 3 | LMR13 | 0.071 | 1 | NA |
| | 0 | 0 | NA | 0 | −1 | con |
| | 0 | 0 | NA | 0 | −1 | infl |
| 24 | 2 | 3 | LMR10 | 0.019 | 1 | NA |
| | 0 | 0 | NA | 0 | −1 | infl |
| | 0 | 0 | NA | 0 | −1 | con |
| 25 | 2 | 3 | LMR8 | 0.442 | 1 | NA |
| | 0 | 0 | NA | 0 | −1 | con |
| | 0 | 0 | NA | 0 | −1 | infl |
| 26 | 2 | 3 | LMR1 | 0.178 | 1 | NA |
| | 0 | 0 | NA | 0 | −1 | infl |
| | 0 | 0 | NA | 0 | −1 | con |
| 27 | 2 | 3 | LMR9 | 0.246 | 1 | NA |
| | 0 | 0 | NA | 0 | −1 | infl |
| | 0 | 0 | NA | 0 | −1 | con |
| 28 | 2 | 3 | LMR7 | 0.407 | 1 | NA |
| | 0 | 0 | NA | 0 | −1 | con |
| | 0 | 0 | NA | 0 | −1 | infl |
| 29 | 2 | 3 | LMR7 | 0.395 | 1 | NA |
| | 0 | 0 | NA | 0 | −1 | con |
| | 0 | 0 | NA | 0 | −1 | infl |
| 30 | 2 | 3 | LMR13 | 0.075 | 1 | NA |
| | 0 | 0 | NA | 0 | −1 | con |
| | 0 | 0 | NA | 0 | −1 | infl |
| 31 | 2 | 3 | LMR14 | 0.065 | 1 | NA |
| | 0 | 0 | NA | 0 | −1 | con |
| | 0 | 0 | NA | 0 | −1 | infl |
| 32 | 2 | 3 | LMR6 | 0.138 | 1 | NA |
| | 0 | 0 | NA | 0 | −1 | con |
| | 4 | 5 | LMR1 | 0.181 | 1 | NA |
| | 0 | 0 | NA | 0 | −1 | infl |
| | 0 | 0 | NA | 0 | −1 | con |
| 33 | 2 | 3 | LMR4 | 0.088 | 1 | NA |
| | 0 | 0 | NA | 0 | −1 | infl |
| | 0 | 0 | NA | 0 | −1 | con |
| 34 | 2 | 3 | LMR8 | 0.446 | 1 | NA |
| | 0 | 0 | NA | 0 | −1 | con |
| | 0 | 0 | NA | 0 | −1 | infl |
| 35 | 2 | 3 | LMR12 | 0.356 | 1 | NA |
| | 0 | 0 | NA | 0 | −1 | infl |
| | 0 | 0 | NA | 0 | −1 | con |
| 36 | 2 | 3 | LMR13 | 0.074 | 1 | NA |
| | 0 | 0 | NA | 0 | −1 | con |
| | 0 | 0 | NA | 0 | −1 | infl |
| 37 | 2 | 3 | LMR12 | 0.358 | 1 | NA |
| | 0 | 0 | NA | 0 | −1 | infl |
| | 0 | 0 | NA | 0 | −1 | con |
| 38 | 2 | 3 | LMR12 | 0.356 | 1 | NA |
| | 0 | 0 | NA | 0 | −1 | infl |
| | 0 | 0 | NA | 0 | −1 | con |
| 39 | 2 | 3 | LMR7 | 0.4 | 1 | NA |
| | 0 | 0 | NA | 0 | −1 | con |
| | 0 | 0 | NA | 0 | −1 | infl |
| 40 | 2 | 3 | LMR11 | 0.164 | 1 | NA |
| | 0 | 0 | NA | 0 | −1 | con |
| | 0 | 0 | NA | 0 | −1 | infl |
| 41 | 2 | 3 | LMR12 | 0.356 | 1 | NA |
| | 0 | 0 | NA | 0 | −1 | infl |
| | 0 | 0 | NA | 0 | −1 | con |
| 42 | 2 | 3 | LMR8 | 0.437 | 1 | NA |
| | 0 | 0 | NA | 0 | −1 | con |
| | 0 | 0 | NA | 0 | −1 | infl |
| 43 | 2 | 3 | LMR3 | 0.122 | 1 | NA |
| | 0 | 0 | NA | 0 | −1 | infl |
| | 0 | 0 | NA | 0 | −1 | con |
| 44 | 2 | 3 | LMR12 | 0.359 | 1 | NA |
| | 0 | 0 | NA | 0 | −1 | infl |
| | 0 | 0 | NA | 0 | −1 | con |
| 45 | 2 | 3 | LMR3 | 0.122 | 1 | NA |
| | 0 | 0 | NA | 0 | −1 | infl |
| | 0 | 0 | NA | 0 | −1 | con |
| 46 | 2 | 3 | LMR9 | 0.235 | 1 | NA |
| | 0 | 0 | NA | 0 | −1 | infl |
| | 0 | 0 | NA | 0 | −1 | con |
| 47 | 2 | 3 | LMR5 | 0.165 | 1 | NA |
| | 0 | 0 | NA | 0 | −1 | con |
| | 4 | 5 | LMR2 | 0.03 | 1 | NA |
| | 0 | 0 | NA | 0 | −1 | con |
| | 0 | 0 | NA | 0 | −1 | infl |
| 48 | 2 | 3 | LMR15 | 0.079 | 1 | NA |
| | 0 | 0 | NA | 0 | −1 | con |
| | 0 | 0 | NA | 0 | −1 | infl |
| 49 | 2 | 3 | LMR9 | 0.246 | 1 | NA |
| | 0 | 0 | NA | 0 | −1 | infl |
| | 0 | 0 | NA | 0 | −1 | con |
| 50 | 2 | 3 | LMR14 | 0.063 | 1 | NA |
| | 0 | 0 | NA | 0 | −1 | con |
| | 0 | 0 | NA | 0 | −1 | infl |
| 51 | 2 | 3 | LMR9 | 0.246 | 1 | NA |
| | 0 | 0 | NA | 0 | −1 | infl |
| | 0 | 0 | NA | 0 | −1 | con |
| 52 | 2 | 3 | LMR4 | 0.122 | 1 | NA |
| | 0 | 0 | NA | 0 | −1 | infl |
| | 0 | 0 | NA | 0 | −1 | con |
| 53 | 2 | 3 | LMR13 | 0.077 | 1 | NA |
| | 0 | 0 | NA | 0 | −1 | con |
| | 4 | 5 | LMR6 | 0.129 | 1 | NA |
| | 0 | 0 | NA | 0 | −1 | con |
| | 0 | 0 | NA | 0 | −1 | infl |
| 54 | 2 | 3 | LMR6 | 0.12 | 1 | NA |
| | 0 | 0 | NA | 0 | −1 | con |
| | 0 | 0 | NA | 0 | −1 | infl |
| 55 | 2 | 3 | LMR11 | 0.168 | 1 | NA |
| | 4 | 5 | LMR13 | 0.1 | 1 | NA |
| | 0 | 0 | NA | 0 | −1 | infl |
| | 0 | 0 | NA | 0 | −1 | con |
| | 0 | 0 | NA | 0 | −1 | infl |
| 56 | 2 | 3 | LMR8 | 0.505 | 1 | NA |
| | 0 | 0 | NA | 0 | −1 | con |
| | 0 | 0 | NA | 0 | −1 | infl |
| 57 | 2 | 3 | LMR6 | 0.12 | 1 | NA |
| | 0 | 0 | NA | 0 | −1 | con |
| | 0 | 0 | NA | 0 | −1 | infl |
| 58 | 2 | 3 | LMR2 | 0.055 | 1 | NA |
| | 0 | 0 | NA | 0 | −1 | con |
| | 4 | 5 | LMR5 | 0.161 | 1 | NA |
| | 0 | 0 | NA | 0 | −1 | con |
| | 0 | 0 | NA | 0 | −1 | infl |
| 59 | 2 | 3 | LMR12 | 0.356 | 1 | NA |
| | 0 | 0 | NA | 0 | −1 | infl |
| | 0 | 0 | NA | 0 | −1 | con |
| | 2 | 3 | LMR9 | 0.235 | 1 | NA |
| | 0 | 0 | NA | 0 | −1 | infl |
| | 0 | 0 | NA | 0 | −1 | con |
| 61 | 2 | 3 | LMR8 | 0.451 | 1 | NA |
| | 0 | 0 | NA | 0 | −1 | con |
| | 4 | 5 | LMR2 | 0.104 | 1 | NA |
| | 0 | 0 | NA | 0 | −1 | infl |
| | 0 | 0 | NA | 0 | −1 | con |
| 62 | 2 | 3 | LMR3 | 0.122 | 1 | NA |
| | 0 | 0 | NA | 0 | −1 | infl |
| | 0 | 0 | NA | 0 | −1 | con |
| 63 | 2 | 3 | LMR15 | 0.079 | 1 | NA |
| | 0 | 0 | NA | 0 | −1 | con |
| | 0 | 0 | NA | 0 | −1 | infl |
| 64 | 2 | 3 | LMR9 | 0.246 | 1 | NA |
| | 0 | 0 | NA | 0 | −1 | infl |
| | 0 | 0 | NA | 0 | −1 | con |
| 65 | 2 | 3 | LMR4 | 0.12 | 1 | NA |
| | 0 | 0 | NA | 0 | −1 | infl |
| | 0 | 0 | NA | 0 | −1 | con |
| 66 | 2 | 3 | LMR1 | 0.174 | 1 | NA |
| | 0 | 0 | NA | 0 | −1 | infl |
| | 0 | 0 | NA | 0 | −1 | con |
| 67 | 2 | 3 | LMR1 | 0.163 | 1 | NA |
| | 0 | 0 | NA | 0 | −1 | infl |
| | 0 | 0 | NA | 0 | −1 | con |
| 68 | 2 | 3 | LMR15 | 0.128 | 1 | NA |
| | 0 | 0 | NA | 0 | −1 | con |
| | 0 | 0 | NA | 0 | −1 | infl |
| 69 | 2 | 3 | LMR6 | 0.129 | 1 | NA |
| | 0 | 0 | NA | 0 | −1 | con |
| | 0 | 0 | NA | 0 | −1 | infl |
| 70 | 2 | 3 | LMR13 | 0.077 | 1 | NA |
| | 0 | 0 | NA | 0 | −1 | con |
| | 0 | 0 | NA | 0 | −1 | infl |
| 71 | 2 | 3 | LMR9 | 0.235 | 1 | NA |
| | 0 | 0 | NA | 0 | −1 | infl |
| | 0 | 0 | NA | 0 | −1 | con |
| 72 | 2 | 3 | LMR9 | 0.246 | 1 | NA |
| | 0 | 0 | NA | 0 | −1 | infl |
| | 0 | 0 | NA | 0 | −1 | con |
| 73 | 2 | 3 | LMR9 | 0.246 | 1 | NA |
| | 0 | 0 | NA | 0 | −1 | infl |
| | 0 | 0 | NA | 0 | −1 | con |
| 74 | 2 | 3 | LMR14 | 0.063 | 1 | NA |
| | 0 | 0 | NA | 0 | −1 | con |
| | 0 | 0 | NA | 0 | −1 | infl |
| 75 | 2 | 3 | LMR4 | 0.12 | 1 | NA |
| | 0 | 0 | NA | 0 | −1 | infl |
| | 0 | 0 | NA | 0 | −1 | con |
| 76 | 2 | 3 | LMR12 | 0.356 | 1 | NA |
| | 0 | 0 | NA | 0 | −1 | infl |
| | 0 | 0 | NA | 0 | −1 | con |
| 77 | 2 | 3 | LMR7 | 0.392 | 1 | NA |
| | 0 | 0 | NA | 0 | −1 | con |
| | 0 | 0 | NA | 0 | −1 | infl |
| 78 | 2 | 3 | LMR5 | 0.171 | 1 | NA |
| | 0 | 0 | NA | 0 | −1 | con |
| | 0 | 0 | NA | 0 | −1 | infl |
| 79 | 2 | 3 | LMR10 | 0.016 | 1 | NA |
| | 0 | 0 | NA | 0 | −1 | infl |
| | 0 | 0 | NA | 0 | −1 | con |
| 80 | 2 | 3 | LMR11 | 0.168 | 1 | NA |
| | 0 | 0 | NA | 0 | −1 | con |
| | 0 | 0 | NA | 0 | −1 | infl |
| 81 | 2 | 3 | LMR11 | 0.168 | 1 | NA |
| | 0 | 0 | NA | 0 | −1 | con |
| | 0 | 0 | NA | 0 | −1 | infl |
| 82 | 2 | 3 | LMR12 | 0.356 | 1 | NA |
| | 0 | 0 | NA | 0 | −1 | infl |
| | 0 | 0 | NA | 0 | −1 | con |
| 83 | 2 | 3 | LMR3 | 0.129 | 1 | NA |
| | 0 | 0 | NA | 0 | −1 | infl |
| | 0 | 0 | NA | 0 | −1 | con |
| 84 | 2 | 3 | LMR9 | 0.235 | 1 | NA |
| | 0 | 0 | NA | 0 | −1 | infl |
| | 0 | 0 | NA | 0 | −1 | con |
| 85 | 2 | 3 | LMR9 | 0.246 | 1 | NA |
| | 0 | 0 | NA | 0 | −1 | infl |
| | 0 | 0 | NA | 0 | −1 | con |
| 86 | 2 | 3 | LMR4 | 0.119 | 1 | NA |
| | 0 | 0 | NA | 0 | −1 | infl |
| | 0 | 0 | NA | 0 | −1 | con |
| 87 | 2 | 3 | LMR15 | 0.094 | 1 | NA |
| | 0 | 0 | NA | 0 | −1 | con |
| | 0 | 0 | NA | 0 | −1 | infl |
| 88 | 2 | 3 | LMR9 | 0.246 | 1 | NA |
| | 0 | 0 | NA | 0 | −1 | infl |
| | 0 | 0 | NA | 0 | −1 | con |
| 89 | 2 | 3 | LMR12 | 0.356 | 1 | NA |
| | 0 | 0 | NA | 0 | −1 | infl |
| | 0 | 0 | NA | 0 | −1 | con |
| 90 | 2 | 3 | LMR10 | 0.019 | 1 | NA |
| | 0 | 0 | NA | 0 | −1 | infl |
| | 0 | 0 | NA | 0 | −1 | con |
| 91 | 2 | 3 | LMR7 | 0.395 | 1 | NA |
| | 0 | 0 | NA | 0 | −1 | con |
| | 0 | 0 | NA | 0 | −1 | infl |
| 92 | 2 | 3 | LMR15 | 0.121 | 1 | NA |
| | 0 | 0 | NA | 0 | −1 | con |
| | 0 | 0 | NA | 0 | −1 | infl |
| 93 | 2 | 3 | LMR8 | 0.446 | 1 | NA |
| | 0 | 0 | NA | 0 | −1 | con |
| | 4 | 5 | LMR3 | 0.129 | 1 | NA |
| | 0 | 0 | NA | 0 | −1 | infl |
| | 0 | 0 | NA | 0 | −1 | con |
| 94 | 2 | 3 | LMR12 | 0.354 | 1 | NA |
| | 0 | 0 | NA | 0 | −1 | infl |
| | 0 | 0 | NA | 0 | −1 | con |
| 96 | 2 | 3 | LMR9 | 0.246 | 1 | NA |
| | 0 | 0 | NA | 0 | −1 | infl |
| | 0 | 0 | NA | 0 | −1 | con |
| 97 | 2 | 3 | LMR5 | 0.165 | 1 | NA |
| | 0 | 0 | NA | 0 | −1 | con |
| | 4 | 5 | LMR15 | 0.077 | 1 | NA |
| | 0 | 0 | NA | 0 | −1 | con |
| | 0 | 0 | NA | 0 | −1 | infl |
| 98 | 2 | 3 | LMR9 | 0.235 | 1 | NA |
| | 0 | 0 | NA | 0 | −1 | infl |
| | 0 | 0 | NA | 0 | −1 | con |
| 99 | 2 | 3 | LMR12 | 0.356 | 1 | NA |
| | 0 | 0 | NA | 0 | −1 | infl |
| | 0 | 0 | NA | 0 | −1 | con |
| 100 | 2 | 3 | LMR12 | 0.356 | 1 | NA |
| | 0 | 0 | NA | 0 | −1 | infl |
| | 0 | 0 | NA | 0 | −1 | con |

1. A method for classifying gut inflammation status of an avian subject or of a group of avian subjects to be tested, an avian test subject, the method comprising:
a. determining a test average methylation level of a panel of pre-selected Low-Methylated Regions (LMRs) in isolated genomic DNA from a gut test sample derived from the avian test subject;
b. comparing the test average methylation level obtained in step (a) with a reference average methylation level of the same panel of LMRs in genomic DNA isolated from at least one avian gut sample having a negative gut inflammation status,
wherein when the test average methylation level is substantially similar to the reference average methylation level, then the test subject has a negative gut inflammation status and when the test average methylation level is different from the reference average methylation level then the test subject has a positive gut inflammation status.

2. The method according to claim 1, wherein the LMRs in the genomic DNA:
have an average methylation ranging from 10% to 50%,
are regions of low CG density;
are enriched for Histone H3 monomethylated at lysine 4 (H3K4me1), DNase I hypersensitive sites (DHSs) and transcriptional coactivators CREB binding protein (CPB) and p300;
are primarily located distal to promoters in intergenic or intronic regions; and
have no single nucleotide polymorphisms (SNPs) in any of the CpG positions.

3. The method according to claim 1, wherein the positive gut inflammation status is further classified into classes of severely inflamed, moderately inflamed, or weakly inflamed based on the average methylation level.

4. The method according to claim 1, wherein the pre-selected panel of LMRs may be identified using at least one machine learning technique.

5. The method according to claim 1, wherein the pre-selected panel of LMRs are identified using at least one machine learning technique called the Random Forest Analysis.

6. The method according to claim 1, wherein the panel of pre-selected LMRs is at least five LMRs selected from the following list of LMRs of LMR1 to LMR15:

| LMR | chromosome | Start | End |
|---|---|---|---|
| 1 | chr1 | 4035842 | 4036031 |
| 2 | chr1 | 194933340 | 194933476 |
| 3 | chr2 | 25436290 | 25437730 |
| 4 | chr2 | 146200788 | 146200966 |
| 5 | chr3 | 27449524 | 27449646 |
| 6 | chr4 | 2434473 | 2434747 |
| 7 | chr5 | 33274718 | 33277003 |
| 8 | chr7 | 2418236 | 2418612 |
| 9 | chr11 | 4103942 | 4104386 |
| 10 | chr12 | 3121599 | 3121777 |
| 11 | chr12 | 16120842 | 16120882 |
| 12 | chr13 | 8094316 | 8094824 |
| 13 | chr13 | 14903544 | 14903714 |
| 14 | chr15 | 7910436 | 7910504 |
| 15 | chr21 | 5370979 | 5371156 |

7. The method according to claim 1, wherein the panel of pre-selected LMRs is at least five LMRs selected from the following list of LMRs of LMR16 to LMR30:

| LMR | chromosome | Start | End |
|---|---|---|---|
| 16 | chr1 | 18054888 | 18054950 |
| 17 | chr1 | 40284190 | 40285196 |
| 18 | chr2 | 29647196 | 29647230 |
| 19 | chr2 | 31991770 | 31991962 |
| 20 | chr2 | 55880962 | 55880988 |
| 21 | chr2 | 121135192 | 121135560 |
| 22 | chr3 | 67444230 | 67444652 |
| 23 | chr3 | 101470002 | 101470054 |
| 24 | chr4 | 2049172 | 2049946 |
| 25 | chr4 | 7321072 | 7321506 |
| 26 | chr6 | 16995134 | 16995161 |
| 27 | chr12 | 1423884 | 1424138 |
| 28 | chr13 | 2182022 | 2182138 |
| 29 | chr14 | 4612818 | 4613149 |
| 30 | chr14 | 12751458 | 12751702 |

8. The method according to claim 1, wherein the average methylation level of the panel of pre-selected LMRs is determined using bisulfite sequencing.

9. The method according to claim 1, wherein the avian subject or the group of avian subjects to be tested is a chicken or a group of chickens.

10. The method according to a claim 1, wherein the gut sample and gut test sample is gut tissue, said gut tissue preferably being ileum or jejunum.

11. The method according to claim 1, wherein the gut test sample is a pooled sample derived from a group of avian subjects to be tested.

12. A method for developing a test system for classifying gut inflammation status of avian gut samples, the method comprising
a. detecting Low-Methylated Regions (LMRs) in genomic DNA in gut sample obtained from an avian subject or from a group of avian subjects with a known gut inflammation status,
b. selecting a panel of LMRs from the LMRs of step (a) suitable for each known gut inflammation status, such that the classification reliability of the selected panel of LMRs is optimized for the assignment of the known gut inflammation status,
c. measuring the average methylation levels of the selected panel of LMRs for each known gut inflammation status, and
d. creating a library of different reference average methylation levels of the selected panel of LMRs for respective known gut inflammation status,
wherein a comparison of an average methylation level obtained from a gut test sample with the reference average methylation levels of the selected panel of LMRs allows for classifying the gut inflammation status of the gut test sample.

13. The method according to claim 12, wherein the LMRs in the genomic DNA:
have an average methylation ranging from 10% to 50%,
are regions of low CG density;
are enriched for Histone H3 monomethylated at lysine 4 (H3K4me1), DNase I hypersensitive sites (DHSs) and transcriptional coactivators CREB binding protein (CPB) and p300;
are primarily located distal to promoters in intergenic or intronic regions; and
have no single nucleotide polymorphisms (SNPs) in any of the CpG positions.

14. The method according to claim 12, wherein the selected panel of LMRs are at least five LMRs selected from the following lists of LMRs of LMR1 to LMR15:

| LMR | chromosome | Start | End |
|---|---|---|---|
| 1 | chr1 | 4035842 | 4036031 |
| 2 | chr1 | 194933340 | 194933476 |
| 3 | chr2 | 25436290 | 25437730 |
| 4 | chr2 | 146200788 | 146200966 |
| 5 | chr3 | 27449524 | 27449646 |
| 6 | chr4 | 2434473 | 2434747 |
| 7 | chr5 | 33274718 | 33277003 |
| 8 | chr7 | 2418236 | 2418612 |
| 9 | chr11 | 4103942 | 4104386 |
| 10 | chr12 | 3121599 | 3121777 |
| 11 | chr12 | 16120842 | 16120882 |
| 12 | chr13 | 8094316 | 8094824 |
| 13 | chr13 | 14903544 | 14903714 |
| 14 | chr15 | 7910436 | 7910504 |
| 15 | chr21 | 5370979 | 5371156 |

15. The method according to claim 12, wherein the selected panel of LMRs are at least five LMRs selected from the following lists of LMRs of LMR16 to LMR30:

| LMR | chromosome | Start | End |
|---|---|---|---|
| 16 | chr1 | 18054888 | 18054950 |
| 17 | chr1 | 40284190 | 40285196 |
| 18 | chr2 | 29647196 | 29647230 |
| 19 | chr2 | 31991770 | 31991962 |
| 20 | chr2 | 55880962 | 55880988 |
| 21 | chr2 | 121135192 | 121135560 |
| 22 | chr3 | 67444230 | 67444652 |
| 23 | chr3 | 101470002 | 101470054 |
| 24 | chr4 | 2049172 | 2049946 |
| 25 | chr4 | 7321072 | 7321506 |
| 26 | chr6 | 16995134 | 16995161 |
| 27 | chr12 | 1423884 | 1424138 |
| 28 | chr13 | 2182022 | 2182138 |
| 29 | chr14 | 4612818 | 4613149 |
| 30 | chr14 | 12751458 | 12751702 |
